(19) United States
(12) Patent Application Publication
(10) Pub. No.: US 2018/0353457 A1
(43) Pub. Date: Dec. 13, 2018

(54) ABIETANE DITERPENOID 7-ACETOXYROYLEANONES FOR USE IN CANCER TREATMENT

(71) Applicant: University of South Carolina, Columbia, SC (US)

(72) Inventors: Ehsan Jabbarzadeh, Columbia, SC (US); Wesley F. Taylor, Cayce, SC (US)

(21) Appl. No.: 16/004,529

(22) Filed: Jun. 11, 2018

Related U.S. Application Data
(60) Provisional application No. 62/628,385, filed on Feb. 9, 2018.
(60) Provisional application No. 62/518,036, filed on Jun. 12, 2017.

(51) Int. Cl. A61K 31/22; A61K 31/7072; A61P 35/00

(52) U.S. Cl. CPC A61K 31/22; A61P 35/00; A61K 31/7072

(57) ABSTRACT

Abietane diterpenoids developed from a 7-acetoxyroyleanone that has been isolated from the root of the plant Salvia leriifolia are utilized in the prevention of growth and development of pathogenic cells, e.g., cancer cells. The 7-acetoxyroyleanones show efficacy in treatment and prevention of a wide range of cancer types as well as other neoplastic diseases as a chemo-preventative, a primary or secondary cytotoxic agent, a sensitizer for other therapies, or one component of a combinatorial treatment.

### CROSS REFERENCE TO RELATED APPLICATION

This application claims filing benefit of U.S. Provisional Patent Application Ser. No. 62/518,036 entitled “Use of a Newly Discovered Abietane Diterpenoid for Cancer Treatment,” having a filing date of Jun. 12, 2017, and claims benefit of U.S. Provisional Patent Application Ser. No. 62/628,385 entitled “Abietane Diterpenoid 7-Acetoxyroyleanone for Use in Cancer Treatment, having a filing date of Feb. 9, 2018, both of which being incorporated herein by reference for all purposes.

### FEDERAL RESEARCH STATEMENT

This invention was made with Government support under Grant No. 1631439, awarded by the National Science Foundation. The Government has certain rights in the invention.

### BACKGROUND

Cancers and other neoplastic disease remain the cause of poor health and early death throughout the world. Despite advances in treatment, cancer remains the second leading cause of death in the United States and is the leading cause of death in 21 states as of 2016. Further, the probability of being diagnosed with an invasive cancer was found to be 42% for men and 37.6% for women living in the United States according to a study by Siegel and others (“Cancer statistics, 2016,” CA Cancer J Clin, vol. 66, no. 1, pp. 7-30, 2016 January-February 2016). The same study reported that 582,623 deaths in 2012 were a result of cancer and projected that 1,685,210 new cancer cases and 595,690 cancer deaths would occur during 2016.

Treatment of cancers has traditionally been accomplished through one of, or a combination of, chemotherapy, surgery, radiotherapy, immunotherapy, and hormone therapy, among others. Unfortunately, differences in genetic expression, drug sensitivity, cell morphology, and metastatic targets across the dozens of known cancer types has stymied the long-term success of treatments. Acquired drug resistance in response to chemotherapy remains another hurdle in cancer treatment that demands a diverse arsenal of cytotoxic agents. Cancer recurrence and distant metastases, potentially explained by inherently robust and drug resistant cancer stem cells, further hinder positive prognoses in cancer patients. It is hypothesized that cancer stem cells can remain dormant and undetected in the body for years before reactivating and beginning the formation of a new tumor. As such, cancer recurrence and distant metastases continue to plague cancer patients after months or even years of remission using current treatments.

The role of immune cells in combatting aggressive tumors has become increasingly recognized in the medical community and has led to new approaches for cancer therapies. FDA approval of the first therapeutic cancer vaccine, sipuleucel-T, and other cancer immunotherapy drugs, including monoclonal antibodies such as ipilimumab, in addition to increased understanding of the immune system's role in the tumor microenvironment, has led to a call for small molecules capable of regulating immune activity and supporting tumor death.

In spite of such advances, many shortcomings and disadvantages of current chemotherapeutics and other cancer treatments are readily apparent. The hair loss, nausea, vomiting, loss of appetite, compromised immune system, and other side effects commonly associated with a cancer therapy are often a consequence of the currently utilized treatments and not the disease itself.

Unfortunately, a push to develop chemotherapeutic drugs capable of targeting a specific molecule or cancer-associated signaling pathway with reduction in side effects has failed to yield the expected improvements in patient prognoses. This is largely due to the ability of cancer cells to utilize a combination of many different cellular mechanisms to enhance viability. In many cases, cancer cells are able to circumvent apoptosis induced from targeted therapies by simply activating other survival pathways after the initial treatment.

Natural products are a historically successful source of medicinally active compounds with fewer unwanted side effects, especially in regard to chemotherapeutics. In fact, 63% of cancer drugs used between 1981 and 2006 were natural products, were inspired by natural products, or were synthesized from a natural pharmacophore. Medicinally active compounds derived from natural materials have the potential to provide targeted cytotoxic and immune modulating responses while limiting the taxing side effects associated with currently utilized cancer treatments. The use of natural products attempts to balance a robust ability to target numerous pathways simultaneously with a historical record of safe human consumption and benign side effects.

There is a need to discover and optimize the use of novel cytotoxic compounds with low IC₅₀ values, diverse biological targets, immune regulatory capability, and diminished side effects. In addition, there is a need for new treatments engineered to target pathogenic cells both during initial treatment of cancers and after remission has occurred in order to prevent cancer recurrence. New treatments based upon natural materials that can provide efficacy with benign or limited side effects would be of great benefit.

### SUMMARY

According to one embodiment, disclosed is a method for inhibiting the growth and development of cancer cells. The method includes delivering a 7-acetoxyroyleanone abietane diterpenoid to an area comprising cancer cells. Beneficially, the method shows efficacy for a large variety of different cancer cell types.

Also disclosed is a composition configured for inhibiting the growth and development of cancer cells that includes a 7-acetoxyroyleanone abietane diterpenoid and a pharmaceutically acceptable carrier.

### BRIEF DESCRIPTION OF THE FIGURES

A full and enabling disclosure of the present subject matter, including the best mode thereof to one of ordinary skill in the art, is set forth more particularly in the remainder of the specification, including reference to the accompanying figures in which:

FIG. 1 illustrates the structure of one embodiment of a 7-acetoxyroyleanone encompassed herein.

FIG. 2 illustrates the Time of Flight mass spectometry (TOF-MS) spectrum (positive ion mode) of the 7-acetoxyroyleanone of FIG. 1.

FIG. 3 illustrates the high resolution mass spectometry (HR-MS) spectrum (positive ion mode) of the 7-acetoxyroyleanone of FIG. 1.

FIG. 4 illustrates the hydrogen nuclear magnetic resonance (¹H-NMR) spectrum of the 7-acetoxyroyleanone of FIG. 1.

FIG. 5 illustrates the carbon 13 nuclear magnetic resonance (¹³C-NMR) spectrum of the 7-acetoxyroyleanone of FIG. 1.

FIG. 6 illustrates the hydrogen correlated spectroscopy (H—H COSY) spectrum of the 7-acetoxyroyleanone of FIG. 1.

FIG. 7 illustrates the heteronuclear single quantum coherence (HSQC) spectrum of the 7-acetoxyroyleanone of FIG. 1.

FIG. 8 illustrates the heteronuclear multiple bond correlation (HMBC) spectrum of the 7-acetoxyroyleanone of FIG. 1 (in DMSO-d6).

FIG. 9 presents the percent viability of MG-63 cancer cells in response to various concentrations of a 7-acetoxyroyleanone after 48 and 72 hours of exposure.

FIG. 10 presents the percent viability of SK-OV-3 cancer cells in response to various concentrations of a 7-acetoxyroyleanone after 48 and 72 hours of exposure.

FIG. 11 presents the percent viability of MDA-MB-231 cancer cells in response to various concentrations of a 7-acetoxyroyleanone after 48 and 72 hours of exposure.

FIG. 12 presents the percent viability of HCT-116 cancer cells in response to various concentrations of a 7-acetoxyroyleanone after 48 and 72 hours of exposure.

FIG. 13 presents the percent viability of HCT 116/200 cancer cells in response to various concentrations of a 7-acetoxyroyleanone after 48 and 72 hours of exposure.

FIG. 14 presents the percent viability of A2789ADR cancer cells in response to various concentrations of a 7-acetoxyroyleanone after 48 and 72 hours of exposure.

FIG. 15 presents the percent viability of HCT 116/200 cancer cells in response to a 48 hour exposure of a 7-acetoxyroyleanone alone and in conjunction with 5-fluoro-2′-deoxyuridine (FdUrd).

FIG. 16 presents the cell number of HCT 116/200 cancer cells in response to a 48 hour exposure of a 7-acetoxyroyleanone alone and in conjunction with 5-fluoro-2′-deoxyuridine (FdUrd).

FIG. 17 presents a waterfall plot of the growth percent of 59 cell lines in response to 10 μM of a 7-acetoxyroyleanone as determined by the NCI-60 one dose screen.

FIG. 18 presents a waterfall plot of the GI50 of 60 cell lines for the 7-acetoxyroyleanone as determined by a NCI-60 five dose screen.

FIG. 19 presents a waterfall plot of the TGI of 60 cell lines for the 7-acetoxyroyleanone as determined by a NCI-60 five dose screen.

FIG. 20 presents a waterfall plot of the LC50 of 60 cell lines for the 7-acetoxyroyleanone as determined by a NCI-60 five dose screen.

FIG. 21 presents dose-response curves of the 60 cell lines examined in an NCI-60 five dose screen; cell lines are grouped by tissue type.

### DETAILED DESCRIPTION

Reference will now be made in detail to various embodiments of the disclosed subject matter, one or more examples of which are set forth below. Each embodiment is provided by way of explanation of the subject matter, not limitation thereof. In fact, it will be apparent to those skilled in the art that various modifications and variations may be made in the present disclosure without departing from the scope or spirit of the subject matter. For instance, features illustrated or described as part of one embodiment, may be used in another embodiment to yield a still further embodiment.

The present disclosure is generally directed to the utilization of abietane diterpenoids that have been found to exhibit efficacy against a large variety of cancer cells. More specifically, disclosed are methods for inhibiting the growth and development of pathogenic cells, and in one particular embodiment of cancer cells, by use of a 7-acetoxyroyleanone abietane diterpenoid. Abietane diterpenoids encompassed herein can generally include those having the following general structure:

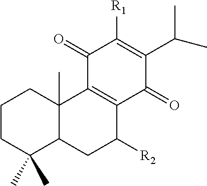

in which R₁ and R₂ are independently selected from —H, C₁₋₁₀ alkyl, C₁₋₁₀ alkoxy, C₁₋₁₀ alkenyl, C₁₋₁₀ alkenoxy, —OH, —OAc, —CHO, -Ph, —OC₆H₅, —OC₆H₄OH, —COC₆H₅, —OCONH₂, —OCONHCH₃, —OCOC₆H₄NH₂, —NH₂, or ═O; and including tautomers of the structures.

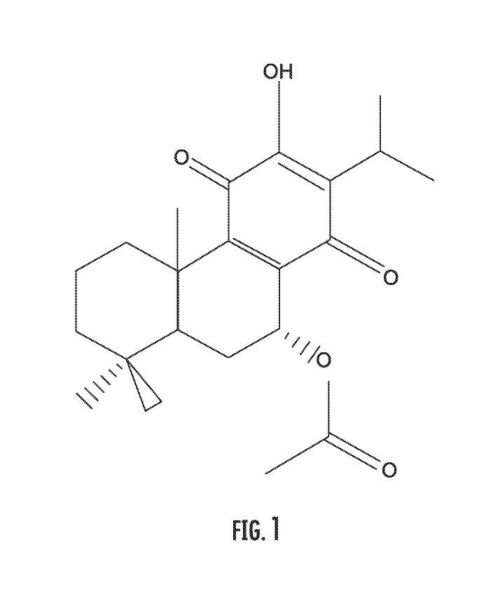

In one embodiment, the methods and compositions can incorporate an abietane diterpenoid as is illustrated in FIG. 1.

7-acetoxyroyleanone abietane diterpenoids encompassed herein have been derived from an abietane diterpenoid that has been isolated from the root of the plant Salvia leriifolia. Salvia leriifolia Benth. (vernacular names include Nuruozak and Jobleh) is a perennial herbaceous plant that grows exclusively in south and tropical regions of certain areas of Iran. Unlike other species of the Salvia genus, the chemical constituents of S. leriifolia are not well recognized. The stem oil of the plant is known to include monoterpenes and sesquiterpenes, and in leaf and flower oils monoterpenes predominate over sesquiterpenes. In recent years, use of this plant in various applications such as the attenuation of morphine dependence, hypoglycemia, as an antinociceptive and antiinflammatory, antioxidant, anti-ischemia, anticonvulsant, antiulcer effects, antibacterial activities and antimutagenic effects have been evaluated (see, e.g., Hosseinzadeh, et al. Iranian Journal of Basic Medical Sciences, 12:1, 2009, 1-8). However, the presently disclosed abietane diterpenoid has not been previously isolated from the root of the plant and the efficacy of this compound isolated from the root of S. leriifolia in inhibition of pathogenic cells has not been previously recognized.

7-acetoxyroyleanone as illustrated in FIG. 1 was isolated from S. leriifolia and purified before being exposed, in vitro, to immortalized tumor cells. The isolated 7-acetoxyroyleanone of S. leriifolia of FIG. 1 as well as the derivatives and tautomers encompassed herein can display efficacy against a broad spectrum of pathogenic cells involved in multiple different types of cancers and other neoplastic disorders. Beneficially, compounds as disclosed herein can exhibit efficacy as a chemo-preventative, a primary or secondary cytotoxic agent, a sensitizer for other therapies, or one component of a combinatorial treatment.

As described further herein, disclosed agents can exhibit anti-proliferative or cytotoxic effects in vitro after about 48 hours of exposure (e.g., about 48 hr. to about 72 hr. of exposure) and have shown efficacy against a number of immortalized cancer cell lines. For example, disclosed materials and methods can be utilized to inhibit growth and development of cancer cells including, but without limitation to, osteosarcoma cells, ovarian adenocarcinoma cells, breast adenocarcinoma cells, colorectal carcinoma cells, as well as pathogenic cells present in neoplastic disorders. Beneficially, the materials can exhibit efficacy against cancer cells exhibiting resistance to other, more traditional chemotherapies such as FdUrd utilized in treatment of colorectal cancer and doxorubicin utilized in treatment of a wide variety of cancers. For example, the materials can inhibit growth and/or development of pathogenic cells including, without limitation, breast cancer cells, bladder cancer cells, Kaposi's sarcoma cells, lymphoma cells, ovarian cancer cells, prostate cancer cells, central nervous system (CNS) cancer cells, renal cancer cells, melanoma cells, colon cancer cells, non-small cell lung cancer cells, and leukemia cells. The disclosed methods and materials can be particularly beneficial against cells that are untreatable through current methods or that have developed a resistance to other, more traditional treatments.

The materials and methods can be targeted in one embodiment against cancer stem cells, which can aid in prevention of cancer metastasis and recurrence.

In another embodiment, disclosed materials can be utilized to target cancer tumors, and can do so without affecting healthy cells, leading to development of treatment protocols having fewer side effects.

As described further in the Examples section below, in vitro toxicity of 7-acetoxyroyleanone has further demonstrated a synergistic relationship with the commonly used chemotherapy drug FdUrd in inducing cell death in colorectal carcinoma cells. As such, the materials can be utilized in one particular embodiment in combination with more traditional chemotherapy and in particular with more traditional colorectal chemotherapy such as FdUrd. Further, the materials can act synergistically with other treatments in clinical use or clinical trials including, but not limited to, surgical resection of tumors, radiation therapy, hormone therapy, immunotherapy, cancer vaccines, etc.

The clinical application and dosage of a 7-acetoxyroyleanone abietane diterpenoid can be tailored to the particular cancer cell of interest as well as to tumor size and stage, patient size, patient medical history, method of delivery, etc., according to methods as are generally known in the art. Use of a compound as disclosed in combination with any number of other bioactive agents, e.g., chemotherapy agents such as FdUrd, has the potential to elicit desirable responses in a large variety of cancer cell types.

By way of example and without limitation, a 7-acetoxyroyleanone as described herein can be utilized in combination with other bioactive agents including classes of antineoplastic drugs including alkaloids/natural products, alkylating agents, antibiotics, antimetabolites, enzymes, farnesyl transferase inhibitors, immunomodulators, immunotoxins, monoclonal antibodies, oligonucleotides, platinum complexes, retinoids, tyrosine kinase inhibitors, androgens, antiadrenals, antiandrogens, antiestrogens, antiprogestins, aromatase inhibitors, estrogens, LH-RH analogs, progestogens, and somatostatin analogs. Examples of common chemotherapeutics on the market that can be utilized in conjunction with 7-acetoxyroyleanone can include, without limitation, the alkaloids paclitaxel, vinblastine, and vincristine; the antimetabolites gemcitabine, 5-fluoruracil, and methotrexate; the antibiotic or antibiotic derivatives doxorubicin, daunorubicin, and bleomycin; the hormonal antineoplastics tamoxifen, diethylstilbestrol, and polyestradiol phosphate; and the immonomodulators sipuleucel-T, interferon-5, and nivolumab.

When utilized in conjunction with another therapy, a 7-acetoxyroyleanone can be administered at the same time as the other therapy, e.g., together in a single composition, or at a different time or on a different schedule, e.g., prior to and/or following administration of a second bioactive agent.

The methods can be utilized in vivo for treatment of cancer or in vitro for study of pathogenic cells or tissue. In order for a 7-acetoxyroyleanone as disclosed to be effectively utilized in a clinical therapy, it can be delivered so as to be provided with suitable bioavailability. According to one treatment method, a composition including a 7-acetoxyroyleanone and a pharmaceutically compatible carrier can be delivered to targeted cells via any pharmaceutically acceptable delivery system. In general, the 7-acetoxyroyleanone may be administered to a subject according to known methods, such as intravenous administration as a bolus or by continuous infusion over a period of time, by intramuscular, intraperitoneal, intracerobrospinal, subcutaneous, intra-articular, intrasynovial, intrathecal, oral, topical, or inhalation routes. Osmotic mini-pumps may also be used to provide controlled delivery of a 7-acetoxyroyleanone through cannulae to the site of interest, such as directly into a metastatic growth. In certain embodiments, a 7-acetoxyroyleanone can be administered directly to the area of a tumor or cancer tissue, including administration directly to the tumor stroma during invasive procedures. A 7-acetoxyroyleanone may also be placed on a solid support such as a sponge or gauze for administration.

Pharmaceutically acceptable carriers include, but are not limited to, saline, buffered saline, glucose in saline, etc. Solid supports, liposomes, nanoparticles, microparticles, nanospheres or microspheres may also be used as carriers for administration of a 7-acetoxyroyleanone. As used herein the term “pharmaceutically acceptable carrier” is intended to include any and all solvents, solubilizers, fillers, stabilizers, binders, absorbents, bases, buffering agents, lubricants, controlled release vehicles, diluents, emulsifying agents, humectants, lubricants, dispersion media, coatings, antibacterial or antifungal agents, isotonic and absorption delaying agents, and the like, compatible with pharmaceutical administration. The use of such media and agents for pharmaceutically active substances is well-known in the art. Except insofar as any conventional media or agent is incompatible with the active compound, use thereof in the compositions is contemplated. Supplementary agents can also be incorporated into the compositions.

The appropriate dosage (“therapeutically effective amount”) of the 7-acetoxyroyleanone can depend, for example, on the severity and course of the cancer, whether the 7-acetoxyroyleanone is administered for therapeutic purposes or in prevention of side effects of a chemotherapy, previous therapy, the patient's clinical history and response to the 7-acetoxyroyleanone, and the discretion of the attending physician, among other factors. A 7-acetoxyroyleanone can be administered to a subject at one time or over a series of treatments and may be administered to the subject at any time.

In one embodiment, a therapeutically effective amount of a 7-acetoxyroyleanone can be in the range of about 0.001 mg/kg body weight/day to about 100 mg/kg body weight/day whether by one or more administrations for instance at a concentration of from about 1 mg/mL to about 50 mg/mL. For example, a 7-acetoxyroyleanone can be administered in an amount of from about 1 mg/kg body weight per day to about 50 mg/kg body weight/day, in some embodiments. For instance, the 7-acetoxyroyleanone can be provided to the targeted site, e.g., a tumor or an in vitro deposit of cancer cells, such that the 7-acetoxyroyleanone is at a concentration of about 10 millimolar (10 mM) or greater at the site of contact, for instance at a concentration of from about 10 mM to about 50 mM in some embodiments. As expected, the dosage will be dependent on the condition, size, age and condition of the patient.

A 7-acetoxyroyleanone may be administered, as appropriate or indicated, in a single dose as a bolus or by continuous infusion, or as multiple doses by bolus or by continuous infusion. Multiple doses may be administered, for example, multiple times per day, once daily, multiple times per week, every 2, 3, 4, 5, 6 or 7 days, weekly, every 2, 3, 4, 5 or 6 weeks or monthly. However, other dosage regimens may be useful. The progress of this therapy is easily monitored by conventional techniques.

It can be advantageous to formulate oral or parenteral compositions in dosage unit form for ease of administration and uniformity of dosage. Dosage unit form as used herein includes physically discrete units suited as unitary dosages for the subject to be treated; each unit may contain a predetermined quantity of active compound calculated to produce the desired therapeutic effect in association with the required pharmaceutical carrier. The specification for the dosage unit forms of the application is dictated by and directly dependent on the unique characteristics of the active compound and the particular therapeutic effect to be achieved, and the limitations inherent in the art of compounding such an active compound for the treatment of individuals.

Pharmaceutical compositions for parenteral, intradermal, or subcutaneous injection can include pharmaceutically acceptable sterile aqueous or nonaqueous solutions, dispersions, suspensions or emulsions as well as sterile powders for reconstitution into sterile injectable solutions or dispersions just prior to use. Examples of suitable aqueous and nonaqueous carriers, diluents, solvents or vehicles include water, ethanol, polyols (e.g., glycerol, propylene glycol, polyethylene glycol and the like), carboxymethylcellulose and suitable mixtures thereof, vegetable oils (e.g., olive oil) and injectable organic esters such as ethyl oleate. A composition can contain minor amounts of auxiliary substances such as wetting or emulsifying agents, pH buffering agents and the like that can enhance the effectiveness of the active ingredient. Proper fluidity may be maintained, for example, by the use of coating materials such as lecithin, by the maintenance of the required particle size in the case of dispersions and by the use of surfactants. A composition may also contain adjuvants such as preservatives, wetting agents, emulsifying agents and dispersing agents. It may also be desirable to include isotonic agents such as sugars, sodium chloride and the like.

For intravenous administration, suitable carriers include, without limitation, physiological saline, bacteriostatic water, Cremophor EL™ (BASF, Parsippany, N.J.) or phosphate buffered saline (PBS). In all cases, an injectable composition should be sterile and should be fluid to the extent that easy syringability exists. It must be stable under the conditions of manufacture and storage and must be preserved against the contaminating action of microorganisms such as bacteria and fungi. Prevention of the action of microorganisms may be ensured by the inclusion of various antibacterial and antifungal agents such as paraben, chlorobutanol, phenol, sorbic acid and the like. Prolonged absorption of the injectable compositions can be brought about by including in the composition an agent that delays absorption, for example, aluminum monostearate or gelatin.

Oral compositions generally include an inert diluent or an edible carrier. They can be enclosed in gelatin capsules or compressed into tablets. For the purpose of oral therapeutic administration, the active compound can be incorporated with excipients and used in the form of tablets, troches, or capsules. Oral compositions can also be prepared using a fluid carrier for use as a mouthwash, wherein the compound in the fluid carrier is applied orally and swished and expectorated or swallowed.

Pharmaceutically compatible binding agents, and/or adjuvant materials can be included as part of an orally ingestible composition. The tablets, pills, capsules, troches and the like can contain any of the following ingredients, or compounds of a similar nature: a binder such as microcrystalline cellulose, gum tragacanth or gelatin; an excipient such as starch or lactose, a disintegrating agent such as alginic acid, Primogel, or corn starch; a lubricant such as magnesium stearate or Stertes; a glidant such as colloidal silicon dioxide; a sweetening agent such as sucrose or saccharin; or a flavoring agent such as peppermint, methyl salicylate, or orange flavoring.

When administered orally in liquid form, a liquid carrier such as water, petroleum, oils of animal or plant origin such as peanut oil, mineral oil, soybean oil, or sesame oil, or synthetic oils may be added. A liquid form may further contain physiological saline solution, dextrose or other saccharide solution, or glycols such as ethylene glycol, propylene glycol or polyethylene glycol. When administered in liquid form, a composition can contain from about 0.5 to 90% by weight of a 7-acetoxyroyleanone (or a mixture thereof), in one embodiment from about 1 to 50% by weight of a 7-acetoxyroyleanone.

For administration by inhalation, the 7-acetoxyroyleanone can be delivered in the form of an aerosol spray from pressured container or dispenser which contains a suitable propellant, e.g., a gas such as carbon dioxide, or a nebulizer.

Systemic administration can also be by transmucosal or transdermal means. For transmucosal or transdermal administration, penetrants appropriate to the barrier to be permeated are used in the formulation. Such penetrants are generally known in the art, and include, for example, for transmucosal administration, detergents, bile salts, and fusidic acid derivatives. Transmucosal administration can be accomplished through the use of nasal sprays or suppositories. For transdermal administration, the pharmaceutical compositions are formulated into ointments, salves, gels, or creams as generally known in the art.

In certain embodiments, a pharmaceutical composition can be formulated for sustained or controlled release of 7-acetoxyroyleanone. Biodegradable, biocompatible polymers can be used, such as ethylene vinyl acetate, polyanhydrides, polyglycolic acid, collagen, polyorthoesters, and polylactic acid. Methods for preparation of such formulations will be apparent to those skilled in the art. The materials can also be obtained commercially. Liposomal suspensions (including liposomes targeted to infected cells with monoclonal antibodies to viral antigens) can also be used as pharmaceutically acceptable carriers. These can be prepared according to methods known to those skilled in the art.

It is to be understood that the in vivo methods have application for both human and veterinary use. The methods of the present invention contemplate single as well as multiple administrations, given either simultaneously or over an extended period of time.

The present disclosure may be better understood with reference to the Example set forth below.

### Example 1

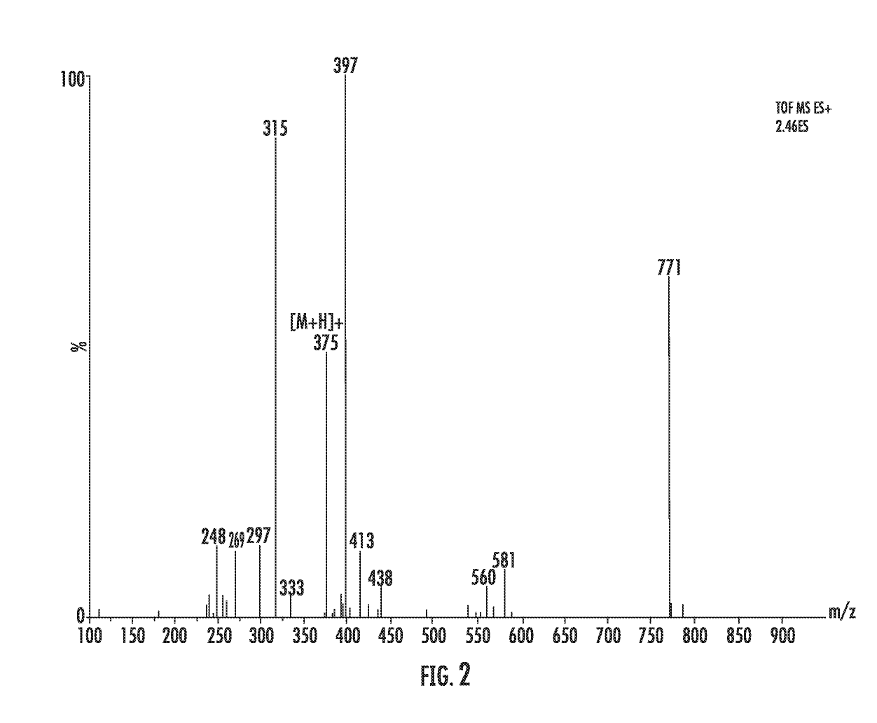

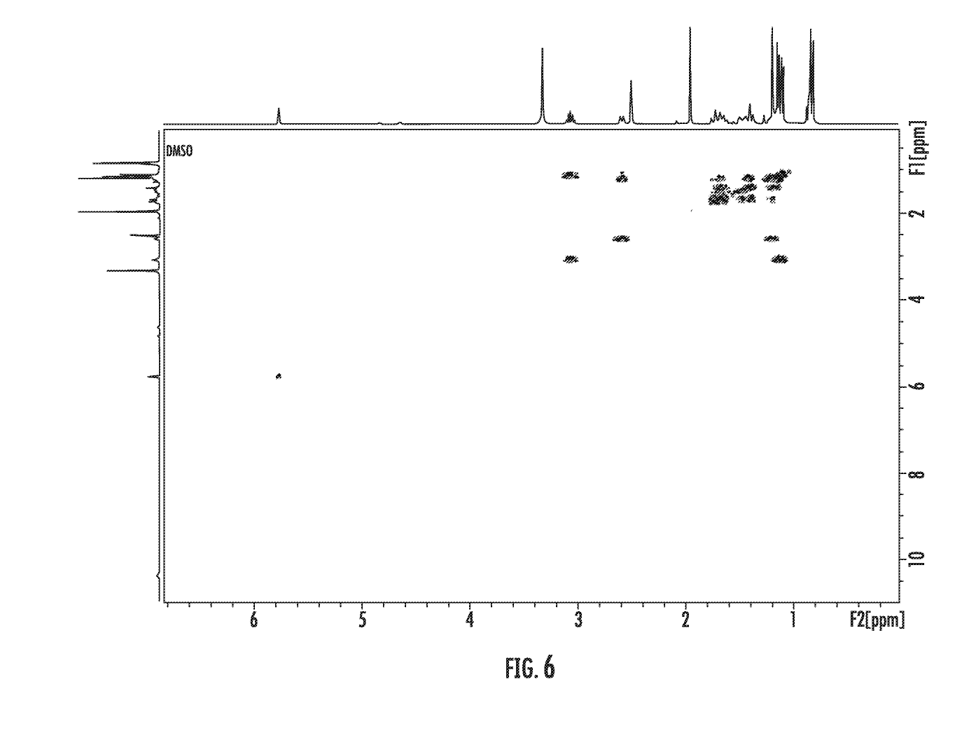

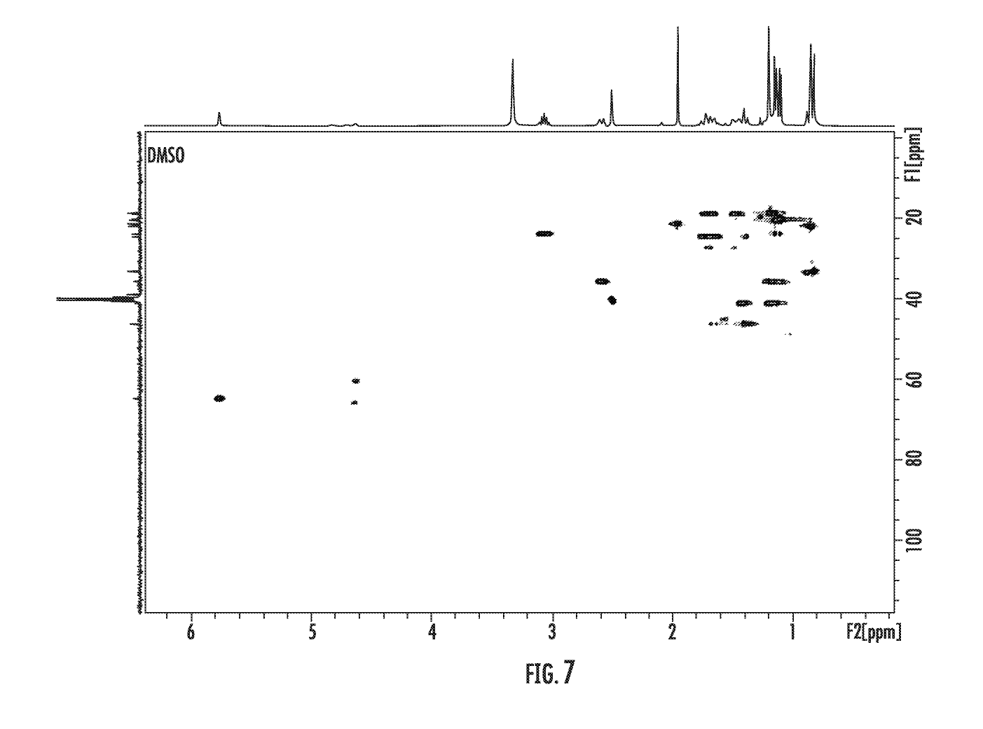

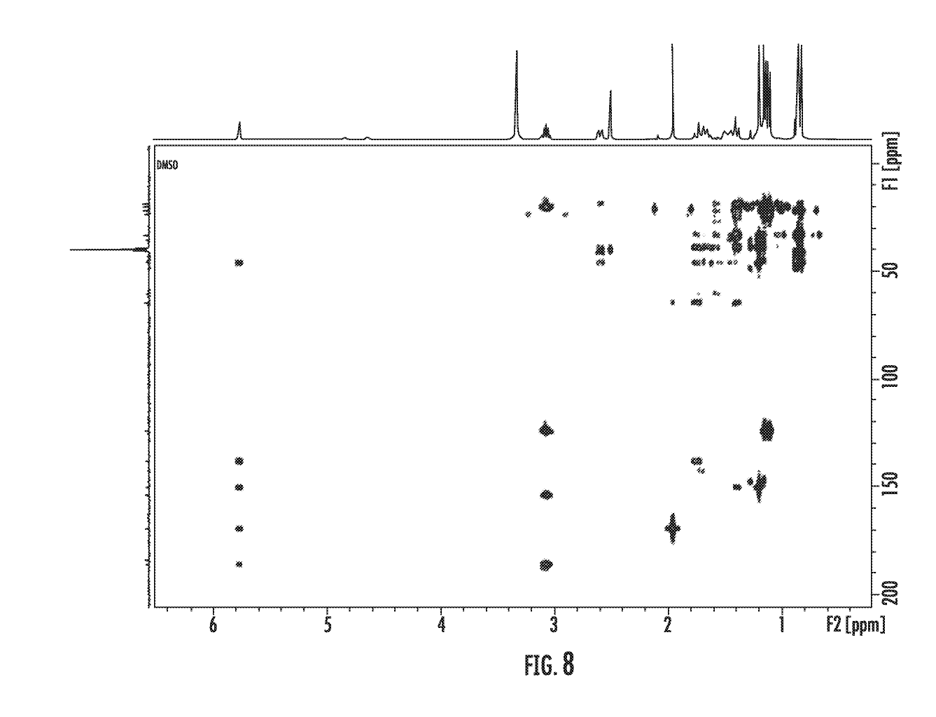

The structure of the 7-acetoxyroyleanone of FIG. 1 was established by means of 1D- and 2D-Nuclear Magnetic Resonance (NMR) (COSY (FIG. 6), HSQC (FIG. 7), and HMBC (FIG. 8)) and the molecular formula was determined by High Resolution Mass Spectrometry (HR-MS) and low resolution Time of Flight Mass Spectrometry (TOF-MS) (FIG. 2). NMR spectra were obtained in DMSO-d6.

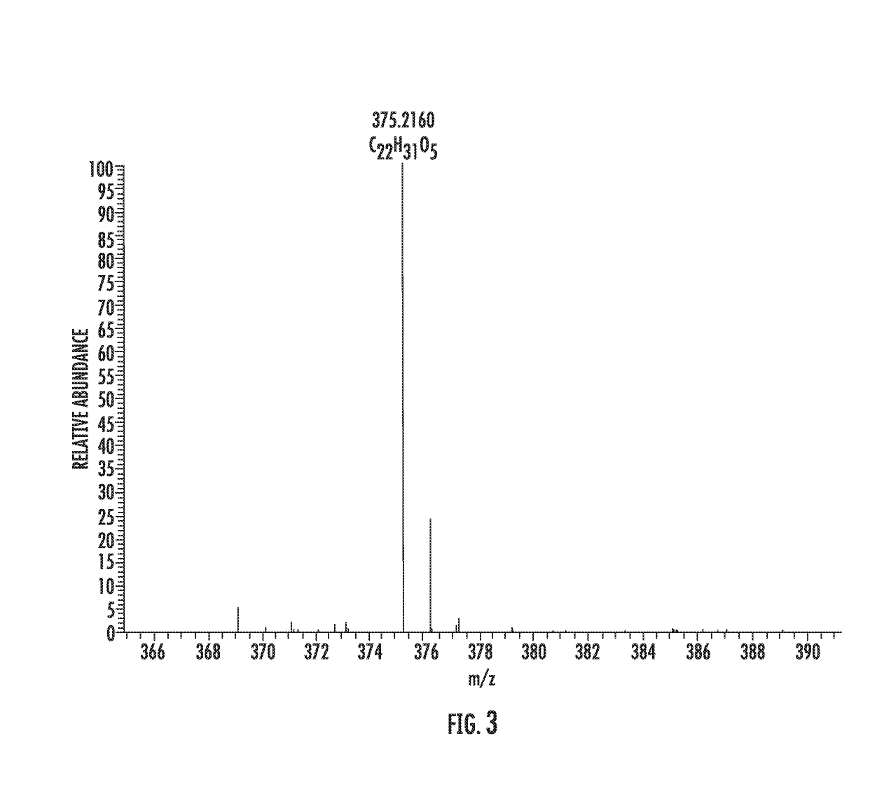

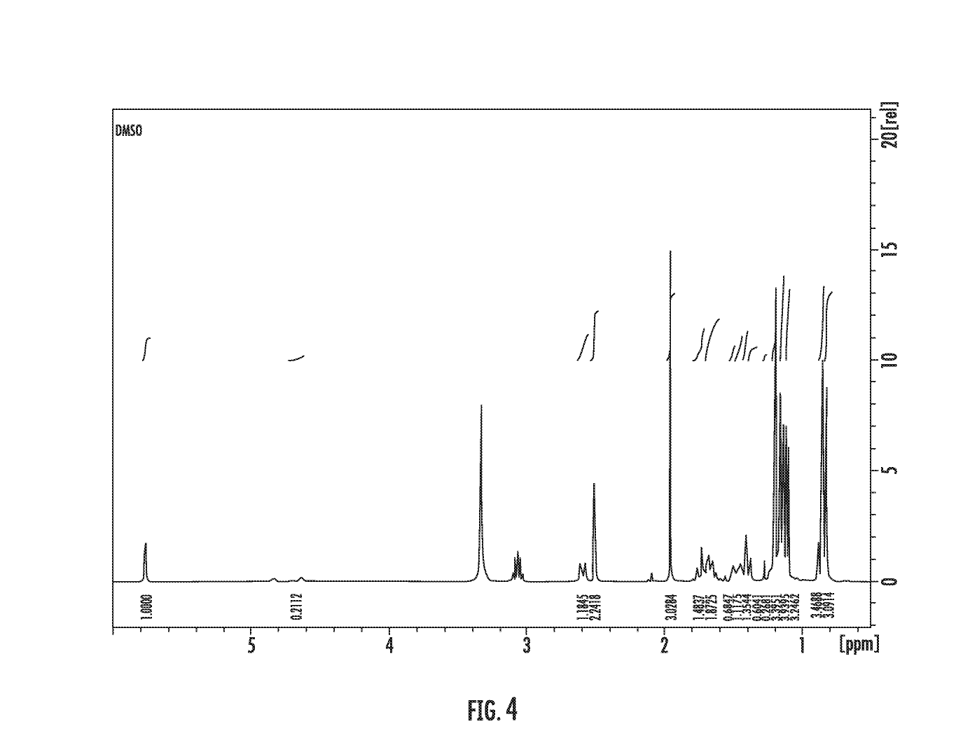

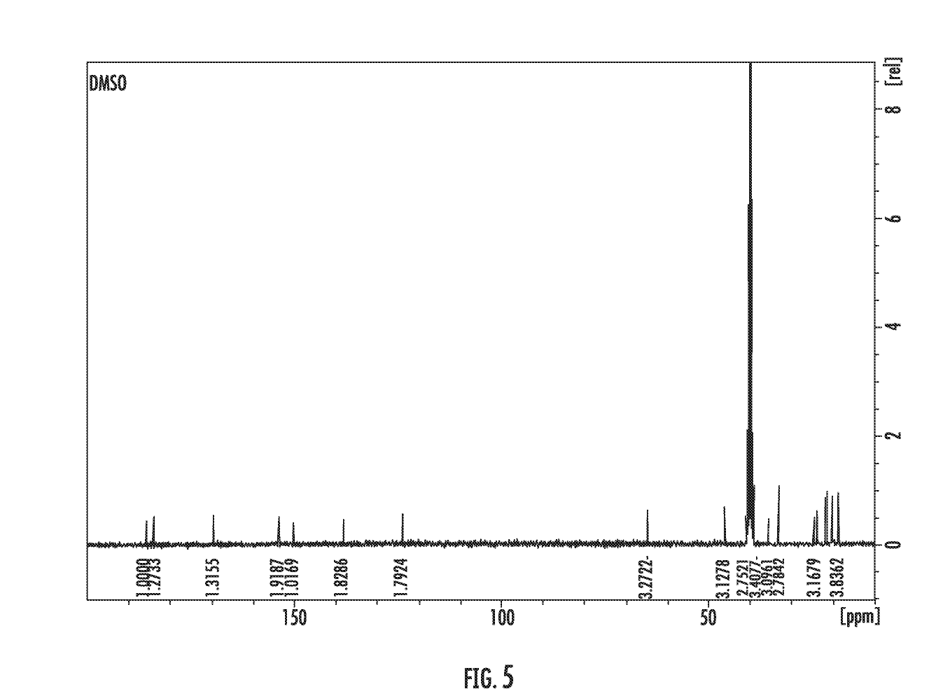

The 7-acetoxyroyleanone was found to have a molecular formula as shown in FIG. 1 of C₂₂H₃₀O₅ (evidenced by HR-MS ([M+H]⁺ ion at m/z 375.2160; FIG. 3)). The NMR spectra (FIG. 4, FIG. 5) confirmed molecular formula of C₂₂H₃₀O₅. Resonance for two tertiary methyls [δ_{H} 0.85 (s, H3-18) and 0.82 (s, H3-19)], two secondary methyls [δ_{H} 1.10 (d, H3-16) and 1.14 (d, H3-17)], and one oxygenated methines [δ_{H} 5.77 (t, H-7)] were observed in ¹H NMR spectrum (FIG. 4). Complementary assignment was obtained through analysis of 2D spectra. The NMR spectra confirmed the 7-acetoxyroyleanone structure.

### Example 2

### Materials and Methods

### Cell Culture

Human cell lines used included MG-63 (osteosarcoma), SK-OV-3 (ovarian adenocarcinoma), MDA-MB-231 (breast adenocarcinoma derived from a metastatic site), HCT 116 (colorectal carcinoma), HCT 116/200 (an FdUrd resistant subclone of HCT 116 cells), and A2780ADR (a doxorubicin resistant subclone of the ovarian carcinoma A2780). All cells were stored in liquid nitrogen until use. MG-63, SK-OV-3, MDA-MB-231, and HCT 116 cell lines were all obtained from ATCC. A2780ADR cells were obtained from Sigma-Aldrich. HCT 116/200 cells were obtained from Dr. Franklin G. Berger from the Center for Colon Cancer Research. MG-63 cells were maintained in MEM medium (Corning) supplemented with 10% Fetal Bovine Essence (VWR) and 1% penicillin/streptomycin solution (Corning). SK-OV-3 cells were maintained in McCoy's 5A Medium (Sigma) supplemented with Fetal Bovine Essence and 1% penicillin/streptomycin. A2780ADR cells were maintained in RPMI 1640 medium (Corning) supplemented with 10% Fetal Bovine Essence and 2 mM L-glutamine (ThermoFisher). MDA-MB-231, HCT 116, and HCT 116/200 cells were all maintained in RPMI 1640 medium supplemented with 10% FBS and 1% penicillin/streptomycin. Cells were incubated at 37° C. and 5% CO₂ throughout the experiment.

### Cytotoxicity Assay

Cells were first grown to approximately 80% confluency within 25 cm³ tissue culture flasks (VWR). The cells were washed once with PBS (Corning) before being typsinized using a 0.25% trypsin, 2.21 mM EDTA, and sodium bicarbonate solution (Corning). Trypsinization was arrested through addition of culture media, and the cell mixtures were centrifuged at 2500 rpm for 5 minutes. Cells were counted using a hemocytometer and their viability was confirmed using trypan blue (Gibco). Cells were then seeded into 96 well tissue culture plates (VWR). MG-63, SK-OV-3, and A2780ADR cells were seeded at a density of 2,000 cells/well with a total volume of 100 μL in each well. MDA-MB-231 cells were seeded at a density of 5,000 cells/well with a total volume of 100 μL in each well. HCT 116 and HCT 116/200 cells were seeded at a density of 4,000 cells/well with a total volume of 100 μL in each well. After seeding, the cells were incubated for 24 hours at 37° C. and 5% CO₂ to allow for cell attachment and renewal of the exponential growth phase. The media was then removed and replaced with media supplemented with the desired concentration of the compound of this invention.

7-acetoxyroyleanone was stored at 4° C. until being first dissolved at 10,000 μg/mL in DMSO and subsequently diluted in culture media to the desired concentration. The vehicle control was cell culture medium supplemented with 0.5% DMSO (Macron Fine Chemicals), representing the highest final concentration of DMSO used to dissolve the natural compound. For comparison, doxorubicin hydrochloride, or DOX (Sigma), and FdUrd (Sigma) supplemented treatments at concentrations sufficient to induce cell death in a majority of cells were also performed.

In combination studies, 7-acetoxyroyleanone and FdUrd were first mixed into the same media and then added to the cells. At either 48 or 72 hours, the media was again removed, the cells were washed with PBS, and media containing 20% MTS solution (Promega) was added to the cells. The cells were incubated for 2 hours, and the absorbance of each well at 490 nm was measured using a Spectramax 190 microplate reader.

### Results

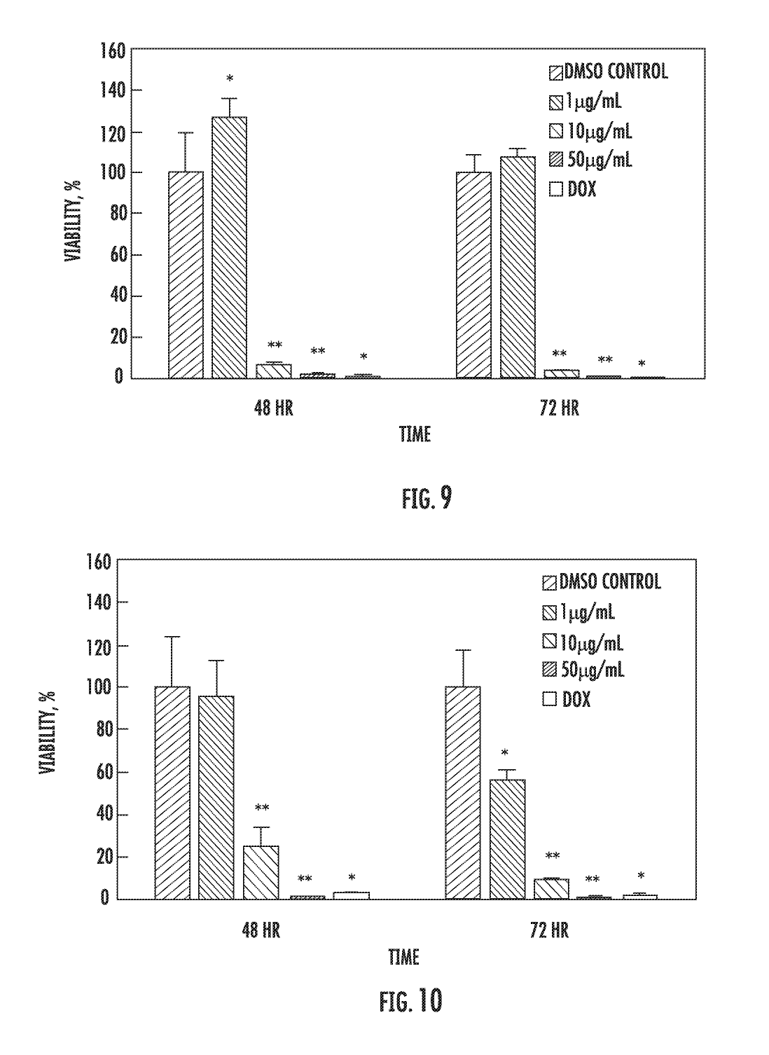

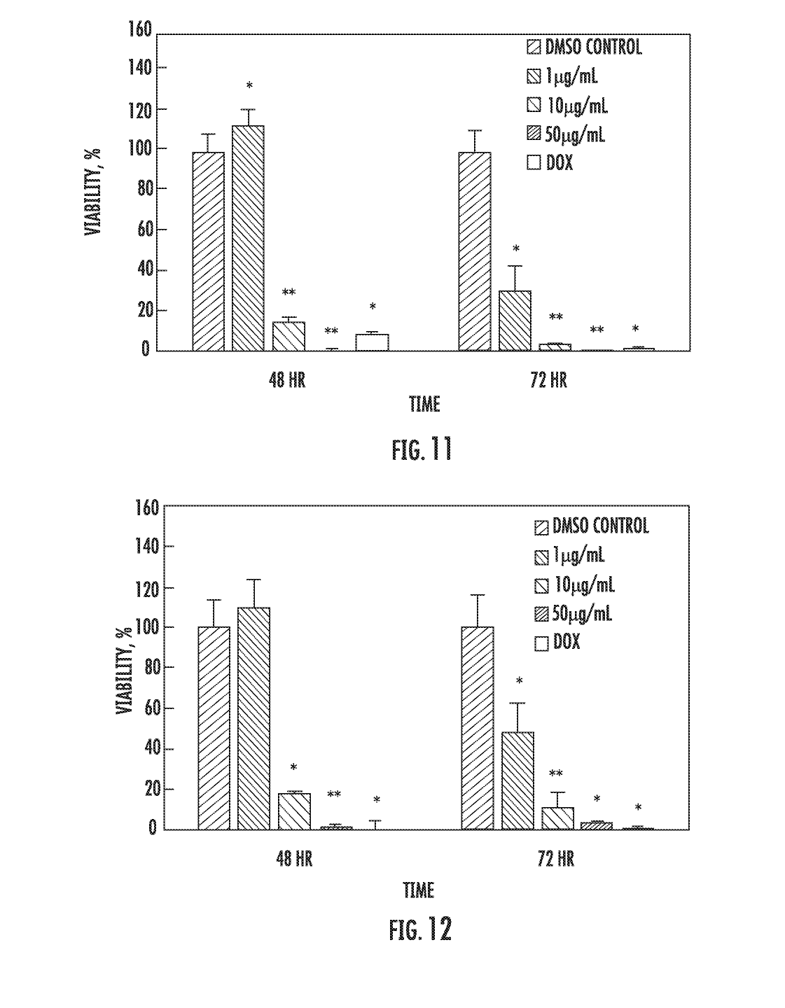

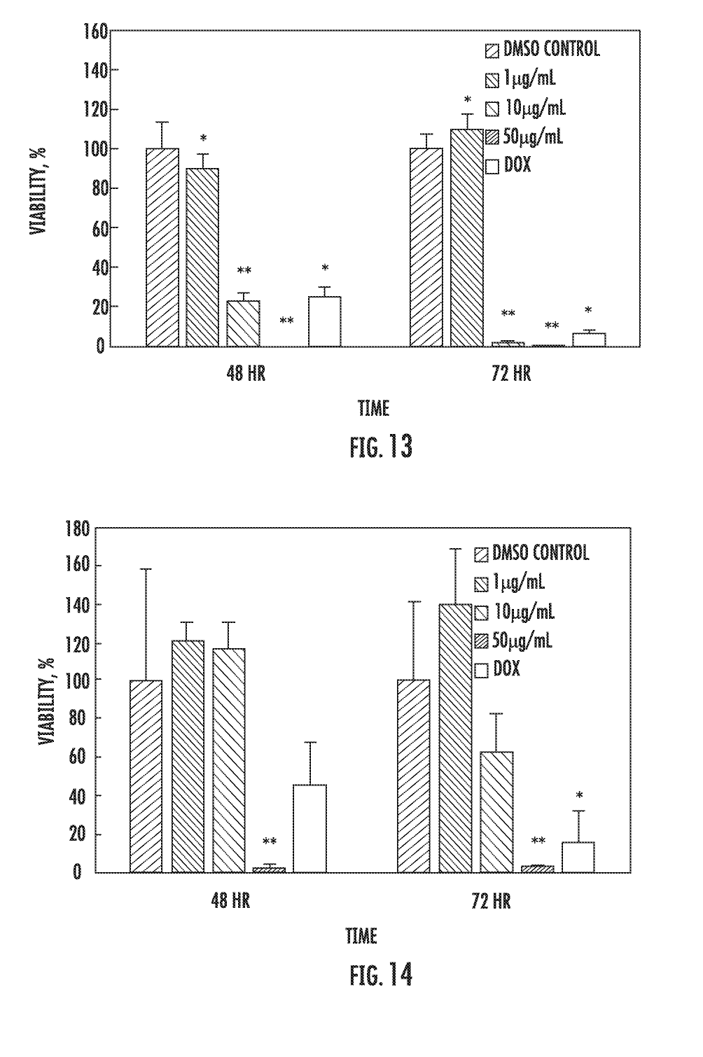

The 7-acetoxyroyleanone showed significant cytotoxic properties against all cell lines tested and at all time points as shown in FIG. 9 (MG-63 cells), FIG. 10 (SK-OV-3 cells, FIG. 11 (MDA-MB-231 cells), FIG. 12 (HCT 116 cells, FIG. 13 (HCT 116/200 cells and FIG. 14 (A2780ADR cells). In FIG. 9-FIG. 14, For all cell lines except A2780ADR (FIG. 14), the concentration of DOX was 1 μg/mL. For the A2780ADR cell line the concentration of DOX was 0.5 μg/m L. (* denotes a significant difference from the control group. ** denotes a significant difference from the previous concentration in addition to the control group. In all cases significance is defined by a two tailed t-test with p<0.05.)

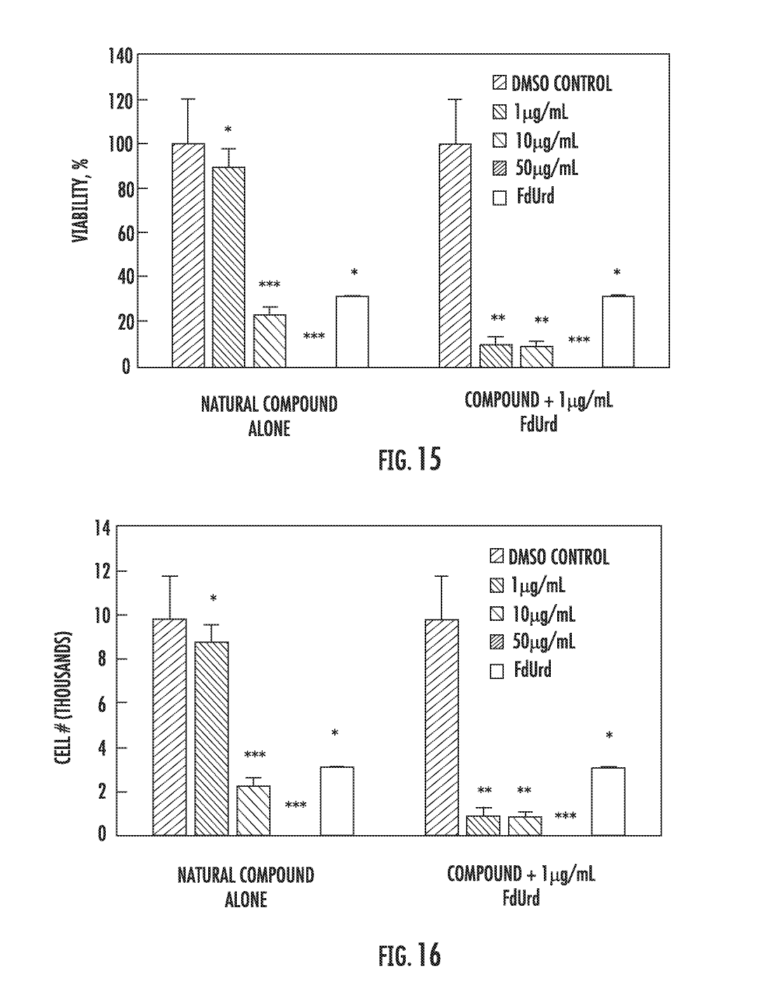

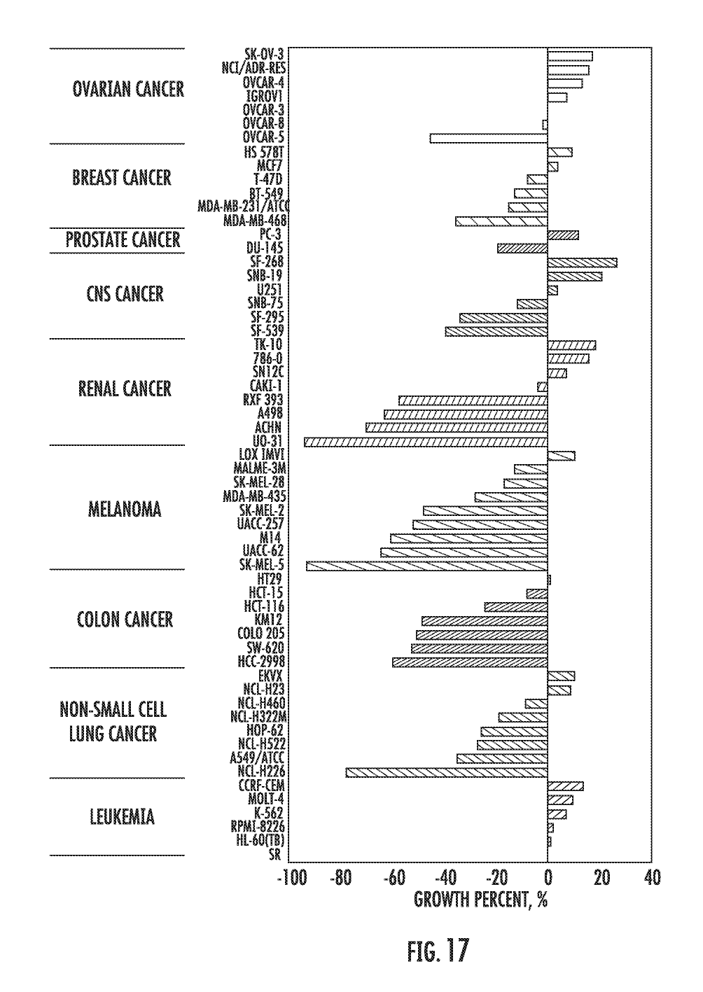

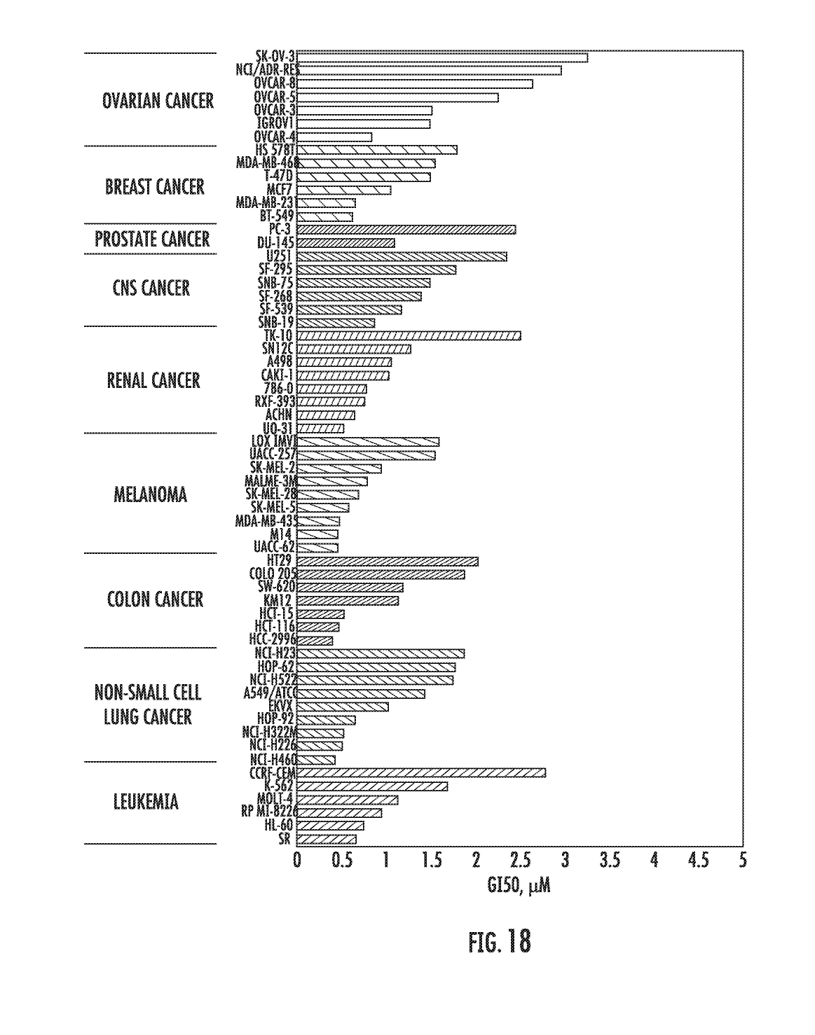

As shown, the effective dose varied from cell line to cell line, but cell death was indicated at the lowest concentration tested (1 μg/mL) in SK-OV-3, MDA-MB-231, and HCT 116 cells. Further, a synergistic cytotoxic effect was observed when the compound was used in combination with FdUrd as seen in FIG. 15 (percent viability) and FIG. 16 (cell number) showing the response of the HCT 116/200 cell line to a 48 hour exposure of various concentrations of 7-acetoxyroyleanone alone and with FdUrd using an MTS assay. (* denotes a viability significantly lower than the control, ** denotes a viability significantly lower than the 1 μg/mL FdUrd treatment, and *** denotes a viability significantly lower than both the 1 μg/mL FdUrd treatment and the 7-acetoxyroyleanone concentration. In all cases significance is defined by a two tailed t-test with p<0.05.)

### Example 3

### Materials and Methods

### NCI60 Cytotoxicity Screening

One dose and five dose cytotoxicity screening was performed by the NCI-60 screening program of the National Institutes of Health (NIH), which is known in the art and available from the NIH. The NCI-60 panel is a collection of cancer cell lines, from various diverse tumors, which was developed in the 1980s by the National Cancer Institute to aid screening efforts for cytotoxic or cytostatic compounds.

In these screens, 60 cell lines, were utilized to determine the cytotoxic effect of small molecules, natural products, and other drug candidates. The cell lines included: ovarian cancer cell lines OVCAR-8, IGROVI, NCl/ADR-RES, OVCAR-5, SK-OV-3, OVCAR-3, OVCAR-4; breast cancer cell lines HS578T, MDA-MB-231/ATCC, MCF7, MDA-MB-468, T-47D, BT-549; prostate cancer cell lines DU-145, PC-3; CNS cancer cell lines U251, SNB-19, SF-539, SF-268, SNB-75; SF-295; renal cancer cell lines TK-10, SN12C, CAKI-1, ACHN, UO-31, RXF 393, A498, 786-0; melanoma cell lines MDA-MB-435, UACC-257, LOX IMVI, SK-MEL-28, M14, SK-MEL-2, MALME-3M, UACC-62, SK-MEL-5; colon cancer cell lines SW-620, KM12, HT29, HCT-15, HCC-2998, COLO 205, HCT-116; non-small cell lung cancer cell lines AS49/ATCC, NCI-H23, HOP-62, NCI-H226, NCI-H322M, NCI-H460, EKVX, HOP-92, NCI-H522; and leukemia cell lines K-562, SR, CCRF-CEM, MOLT-4, HL-60(TB), RPMI-8226.

For the one dose assay, each cell line was treated with 10 μM of the 7-acetoxyroyleanone of FIG. 1 to determine cytotoxicity. The five dose screening was performed with four 10-fold dilutions of the maximum drug concentration examined, 100 μM. Each tumor cell line was maintained in RPMI 1640 media supplemented with 5% fetal bovine serum and 2 mM L-glutamine. The cells were then seeded into 96 well plates at a cell density ranging from 5,000 to 40,000 cells per well depending on the cell line (each well containing 100 μL of cell suspension). The cells were then incubated for 24 hours at 37° C., 5% CO₂, and 100% relative humidity to allow the cells to attach (if they were attached cell lines) and resume their growth phase. Two plates of each cell line which had not been exposed to the 7-acetoxyroyleanone were then fixed with TCA at the time when the 7-acetoxyroyleanone was added to the remaining plates. The remaining plates of the one dose screen were treated with 100 μL of a dilution of 7-acetoxyroyleanone which had been made by diluting the compound in DMSO to 400 times the experimental concentration and subsequently diluting the solution in culture media supplemented with 50 μg/mL gentamycin to twice the experimental concentration.

The five dose screen was performed by creating four 10-fold dilutions of the highest concentration of the diluted 7-acetoxyroyleanone made in the same manner as the one dose screen. The serial dilutions were added to different experimental wells (100 μL per well). A control for each cell type was also simultaneously created in both screens which was treated with 100 μL of culture media without 7-acetoxyroyleanone. The cells were then incubated for 48 hours at 37° C., 5% CO₂, and 100% relative humidity. In the case of the attached cell lines, the cells were then fixed in situ by the gentle addition of cold TCA (10% final TCA concentration) and incubated for 1 hour at 4° C. The plates were washed with tap water five times and air dried. The fixed cells were then incubated for 10 minutes with 100 μL of 0.4% sulforhodamine B (SRB) in 1% acetic acid. The supernatant was removed, and the plates were washed five times with 1% acetic acid and air dried. The bound stain was then dissolved in 10 mM trizma base and the absorbance was read using an automated plate reader at 515 nm.

Suspension cells were treated in the same fashion with the exception that the cells were fixed once they had settled at the bottom of the wells using 50 μL of 80% TCA. The absorbance of each well was then converted to growth percent using equation 1 (below) where Tᵢ is the average absorbance of the samples treated with a certain concentration of the compound of this invention, T_{z} represents the average absorbance of the samples fixed at the time the compound of interest was added to the experimental groups, and C is the average absorbance of the media treated control.

$$Growth Percent = \{ \begin{matrix}\frac{{T}_{i} - {T}_{z}}{C - {T}_{z}} * 100 , & for {T}_{i} \geq {T}_{z} \\ \frac{{T}_{i} - {T}_{z}}{{T}_{z}} * 100 , & for {T}_{i} < {T}_{z}\end{matrix} \qquad ( 1 )$$

Using this equation, the GI50, TGI, and LC50 of the compound was determined for each cell line as the concentration that resulted in the growth percent equaling 50%, 0%, and −50%, respectively.

### Compare Analysis

Based upon the GI50, TGI, and LC50 concentration profile across all of the cell lines examined, the 7-acetoxyroyleanone was compared to existing cytotoxic agents. The COMPARE algorithm developed for the NCI60 screen determined a Pearson correlation coefficient (PCC) between the 7-acetoxyroyleanone and each compound within the desired database. Based upon the mechanism of action of the highly correlated compounds, the mechanism of action of the 7-acetoxyroyleanone can be determined. In this case, the 7-acetoxyroyleanone was analyzed against the standard agents database, a set of FDA approved or promising anti-cancer drugs. The COMPARE analysis was performed for the GI50 profile, the TGI profile, and the LC50 profile. The minimum PCC reported was 0.5, the minimum number of common cell lines required was 40, and the minimum standard deviation allowed was 0.05. If numerous compounds with a PCC higher than 0.5 were found, the top 10 unique results were reported.

### Results and Discussion

The cytotoxicity of the 7-acetoxyroyleanone was demonstrated by the NCI60 screens. In the one dose screen, 10 μM of the 7-acetoxyroyleanone appeared to be above the TGI concentration in at least one example of each cancer type screened as shown in FIG. 17. Further, the growth of each cell line tested in the NCI-60 one dose screen was inhibited by at least 72.88% compared to the control. On average for all the cell types screened, 10 μM of 7-acetoxyroyleanone resulted in −18.5 growth percent, indicating that 7-acetoxyroyleanone induced cell death in a wide variety of cancer types. The one dose screen further indicated that 7-acetoxyroyleanone had a range of effectiveness within each tissue subdivision and that 7-acetoxyroyleanone induced a large percentage of cell death in select cell lines from the renal cancer, melanoma, colon cancer, and non-small cell lung cancer subdivisions. 7-acetoxyroyleanone did not appear to induce cell death, but rather growth inhibition in each of the leukemia cell lines. Taken together, these results indicate that 7-acetoxyroyleanone selectively targeted certain cell lines across a variety of tissues.

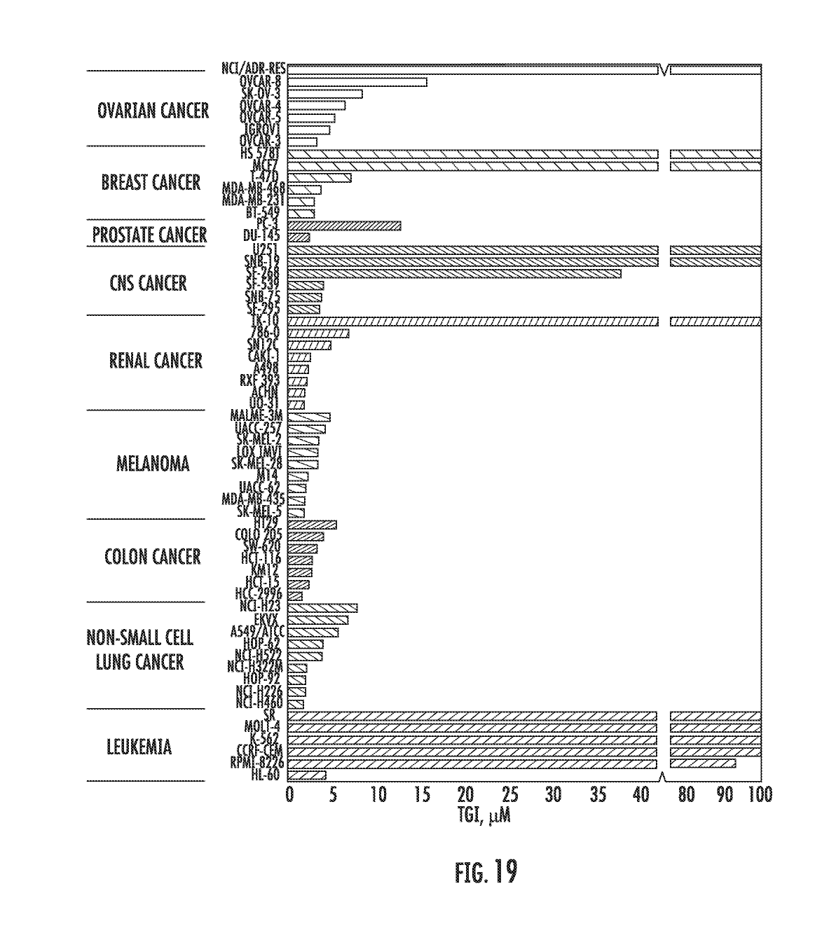

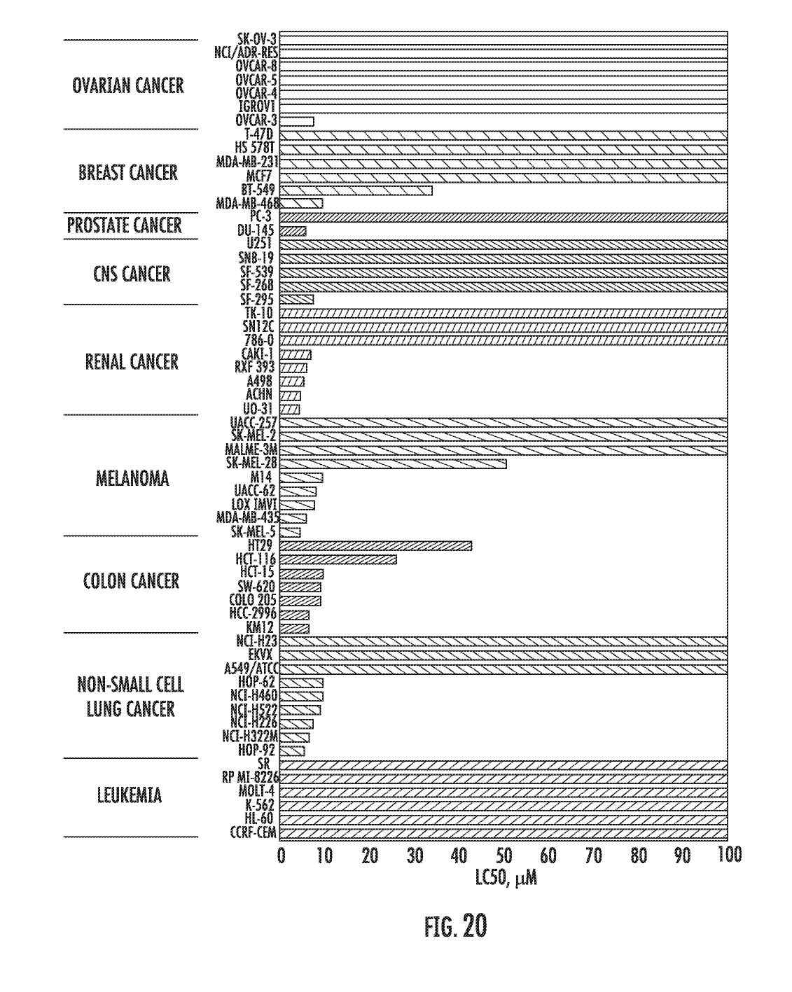

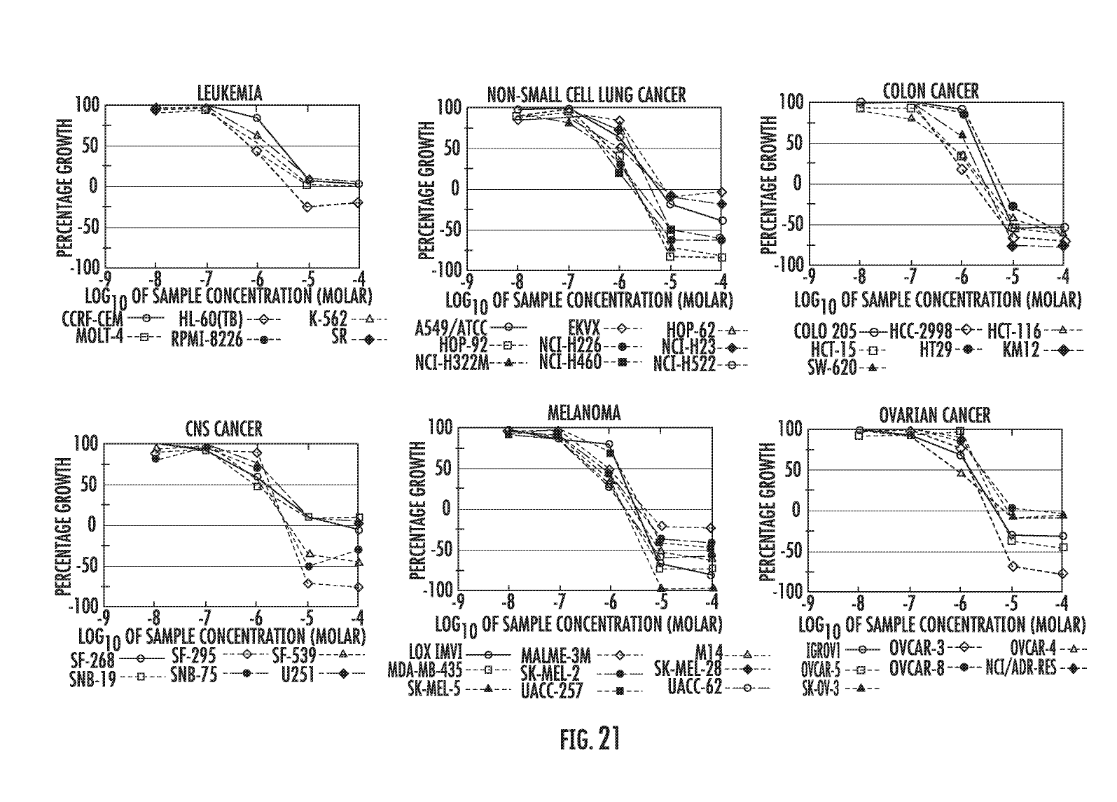

The NCI-60 five dose screen was performed based upon the positive results from the one dose screen. Waterfall plots of the determined GI50, TGI, and LC50 concentrations are displayed in FIG. 18, FIG. 19, and FIG. 20, respectively. The average GI50 concentration was found to be 1.27 μM across all 60 cell lines examined (FIG. 18). The average TGI concentration was found to be 6.69 μM across the 50 cell lines for which the TGI concentration was within the range tested (FIG. 19). The average LC50 concentration was found to be 11.5 μM across the 29 cell lines for which the LC50 concentration was within the range tested (FIG. 20). The range of the GI50 concentrations across all 60 cell lines was only 2.86 μM, supporting the global and potent growth inhibitory capability of the compound. No significant difference was readily apparent between the GI50 concentrations of each cancer tissue subtype.

The selectivity of 7-acetoxyroyleanone became more apparent when examining the TGI and LC50 concentrations of the cell lines. The TGI concentrations of 56 of the 60 cell lines examined were either less than 10 μM or had not been reached at the highest concentration screened, 100 μM. Similarly, the LC50 concentrations of 55 of the 60 cell lines examined were either less than 10 μM or had not been reached at the highest concentration screened, 100 μM. Additionally, at least one cell line from each tissue type had a TGI concentration less than 10 μM, and at least one cell line from each tissue type except leukemia had a LC50 concentration less than 10 μM. These results, when taken together with the GI50 data, show the ability of 7-acetoxyroyleanone to either induce potent cell death in cell lines or arrest cell growth with minimal cytotoxicity. This selectivity is crucial to minimizing the side effects associated with most anti-cancer drugs while remaining effective against a tumor.

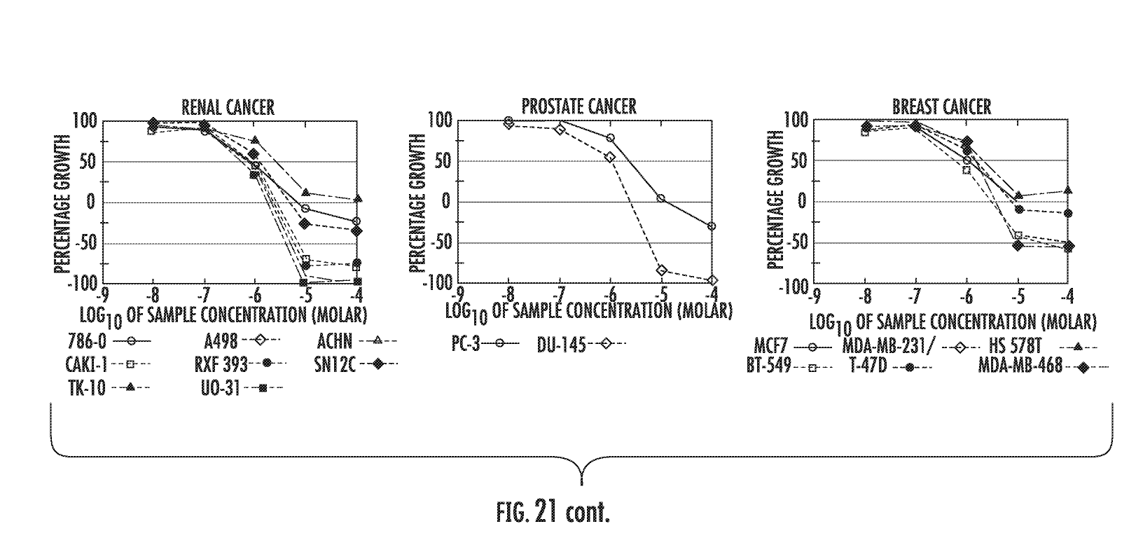

The most sensitive cell line to the growth inhibition caused by 7-acetoxyroyleanone was the colon cancer cell line HCC-2996 with GI50 and TGI concentrations of 0.405 μM and 1.62 μM respectively. The most sensitive cell line to the cell death inducing ability of 7-acetoxyroyleanone was the melanoma cell line SK-OV-3 with a LC50 concentration of 4.34 μM. Interestingly, the LC50 of each of the leukemia cell lines had not been reached at the highest concentration of 7-acetoxyroyleanone tested, 100 μM, suggesting that 7-acetoxyroyleanone may have lower direct effect against leukemic cancers. Further insights can be gleaned from the dose the dose-response curve for each of the 60 cell lines examined in the NCI60 five dose screen (FIG. 21). From this figure in addition to the results from FIG. 18-20, it is clear that 7-acetoxyroyleanone inhibits cell growth or is cytotoxic to a broad spectrum of cancer types. FIG. 21 also shows a number of cell lines, most notably those derived from leukemia, whose dose response curve asymptotically approach 0% growth as the concentration of 7-acetoxyroyleanone was increased. This type of dose response behavior further supports the hypothesis that while the growth inhibitory properties of 7-acetoxyroyleanone affects nearly all cells equally, cell death occurs in a select number of cell lines.

Using the GI50, TGI, and LC50 profiles determined from the NCI-60 screen, a COMPARE analysis was performed against the standard agents database. No compound was found for which a PCC greater than 0.5 existed between the GI50 concentrations of that compound and the 7-acetoxyroyleanone. The compounds which had a PCC greater than 0.5 between their TGI or LC50 profiles and the profiles of 7-acetoxyroyleanone are shown in Table 1.

TABLE 1

| TGI Correlation | | |
|---|---|---|
| Target Compound | PCC | Mechanism of Action |
| 5-azacytidine | 0.572 | antimetabolite |
| dihydrolenperone | 0.524 | — |
| aclacinomycin A | 0.519 | anthracycline/topoisomerase inhibitor |
| teroxirone | 0.514 | DNA alkylating agent |
| DUP785 (brequinar) | 0.507 | antimetabolite disruptor of pyrimidine |
| | | biosynthesis |
| mithramycin | 0.501 | DNA damaging agent |
| LC50 Correlation | | |
| Target Compound | PCC | Mechanism of Action |
| cyclodisone | 0.587 | DNA alkylating agent |
| tetrocarcin A sodium | 0.548 | RNA synthesis inhibitor |
| salt | | |
| DUP785 (brequinar) | 0.516 | antimetabolite disruptor of pyrimidine |
| | | biosynthesis |
| rapamycin | 0.514 | mTOR inhibitor |
| topotecan | 0.512 | topoisomerase inhibitor |

The mechanism of action of the reported compounds nearly exclusively related to DNA damage or inhibition of DNA synthesis. As a result, it is likely that 7-acetoxyroyleanone imparts it cytostatic and cytotoxic effects by damaging or interfering with the synthesis of DNA. While the moderate correlations reported in Table 1 do give insight to the potential mechanism of action of 7-acetoxyroyleanone, the lack of strong correlations (PCC≥0.6) may suggest that the molecular target of 7-acetoxyroyleanone is unique. For the cell lines in which 7-acetoxyroyleanone induced strong cell death, the LC50 concentration of 7-acetoxyroyleanone was competitive with the LC50 concentrations of FDA approved DNA influencing compounds. For example, Table 2 below shows the comparison of the LC50 concentration of 7-acetoxyroyleanone compared to a commonly used topoisomerase poison, doxorubicin. The high level of selectivity demonstrated by 7-acetoxyroyleanone combined with the potent cell death inducing potential makes it an attractive molecule for the treatment of cancer.

TABLE 2

| | | Compound | Doxorubicin |
|---|---|---|---|
| Cell Line | Cancer Subtype | LC50, μM | LC50, μM |
| OVCAR-3 | Ovarian | 7.46 | 23.4 |
| DU-145 | Prostate | 5.67 | 23.4 |
| SF-295 | CNS | 7.37 | 19.1 |
| MDA-MB-468 | Breast | 9.46 | 2.45 |
| A498 | Renal | 5.19 | 3.72 |
| RXF 393 | Renal | 5.87 | 4.79 |
| SK-MEL-5 | Melanoma | 4.34 | 1.62 |
| MDA-MB-435 | Melanoma | 5.87 | 2.88 |
| COLO 205 | Colon | 6.3 | 4.90 |
| HCC-2996 | Colon | 6.41 | 6.76 |
| HOP-92 | Non-small Cell Lung | 5.4 | 20.9 |
| | Cancer | | |
| NCI-H522 | Non-small Cell Lung | 9.02 | 9.33 |
| | Cancer | | |

While certain embodiments of the disclosed subject matter have been described using specific terms, such description is for illustrative purposes only, and it is to be understood that changes and variations may be made without departing from the spirit or scope of the subject matter.

What is claimed is:

1. A method for inhibiting growth and development of cancer cells, the method comprising delivering to an area comprising cancer cells a 7-acetoxyroyleanone having the following structure: 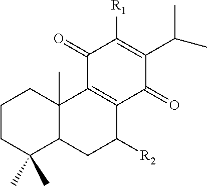
or a tautomer thereof, in which R₁ and R₂ are independently selected from —H, C₁₋₁₀ alkyl, C₁₋₁₀ alkoxy, C₁₋₁₀ alkenyl, C₁₋₁₀ alkenoxy, —OH, —OAc, —CHO, -Ph, —OC₆H₅, —OC₆H₄OH, —COC₆H₅, —OCONH₂, —OCONHCH₃, —OCOC₆H₄NH₂, —NH₂, or ═O.

2. The method of claim 1, wherein the 7-acetoxyroyleanone is delivered to the cancer cells such that the 7-acetoxyroyleanone contacts the cancer cells at a concentration of about 10 millimolar or greater.

3. The method of claim 1, wherein the 7-acetoxyroyleanone is delivered to the cancer cells such that the 7-acetoxyroyleanone contacts the cancer cells at a concentration of from about 10 millimolar to about 50 millimolar.

4. The method of claim 1, wherein the 7-acetoxyroyleanone is delivered to the area in multiple doses.

5. The method of claim 1, wherein the method is an in vitro method.

6. The method of claim 1, wherein the cancer cells comprise breast cancer cells, bladder cancer cells, Kaposi's sarcoma cells, lymphoma cells, ovarian cancer cells, prostate cancer cells, central nervous system cancer cells, renal cancer cells, melanoma cells, colon cancer cells, non-small cell lung cancer cells, or leukemia cells.

7. The method of claim 1, the method comprising delivering the 7-acetoxyroyleanone to the cancer cells in conjunction with a second bioactive agent.

8. The method of claim 7, the method comprising delivering the 7-acetoxyroyleanone and the second bioactive agent to the cancer cells together in a single composition.

9. The method of claim 7, wherein the 7-acetoxyroyleanone is delivered separately from the delivery of the second bioactive agent to the area.

10. The method of claim 7, wherein the 7-acetoxyroyleanone is delivered prior to delivery of the second bioactive agent to the area.

11. The method of claim 7, wherein the 7-acetoxyroyleanone is delivered following delivery of the second bioactive agent to the area.

12. The method of claim 7, wherein the second bioactive agent comprises a chemotherapy agent.

13. The method of claim 12, wherein the chemotherapy agent comprises 5-fluoro-2′-deoxyuridine.

14. The method of claim 1, wherein the cancer cells are resistant to 5-fluoro-2′-deoxyuridine.

15. The method of claim 1, wherein the cancer cells are resistant to doxorubicin.

16. A composition comprising a 7-acetoxyroyleanone having the following structure: 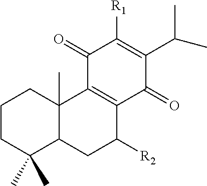
or a tautomer thereof, in which R₁ and R₂ are independently selected from —H, C₁₋₁₀ alkyl, C₁₋₁₀ alkoxy, C₁₋₁₀ alkenyl, C₁₋₁₀ alkenoxy, —OH, —OAc, —CHO, -Ph, —OC₆H₅, —OC₆H₄OH, —COC₆H₅, —OCONH₂, —OCONHCH₃, —OCOC₆H₄NH₂, —NH₂, or ═O and a pharmaceutically compatible carrier.

17. The composition of claim 16, further comprising a second bioactive agent.

18. The composition of claim 17, wherein the second bioactive agent is a chemotherapy agent.

19. The composition of claim 18, wherein the chemotherapy agent exhibits activity against breast cancer cells, bladder cancer cells, Kaposi's sarcoma cells, lymphoma cells, or leukemia cells.

20. The composition of claim 16, wherein the composition is configured for in vivo delivery.
